(19) United States
(12) Patent Application Publication
(10) Pub. No.: US 2024/0290474 A1
(43) Pub. Date: Aug. 29, 2024

(54) SENTIMENT-BASED ANALYTICS MANAGEMENT SYSTEMS AND METHODS

(71) Applicant: Optum, Inc., Minnetonka, MN (US)

(72) Inventors: Christopher Mayer, Johns Creek, GA (US); Sunay Shah, Hopewell, NJ (US); Michael Arnold Lardinois, Cottage Grove, WI (US); Balaji Lakshmi Ramakrishnan, Cumming, GA (US); James McCarter Taylor, Roswell, GA (US); Sarah Paik, Chicago, IL (US); Jessie Lilly, Augusta, ME (US)

(21) Appl. No.: 18/173,319

(22) Filed: Feb. 23, 2023

(51) Int. Cl. G16H 40/20; G06N 20/00; G06Q 30/0282

(52) U.S. Cl. CPC G16H 40/20; G06N 20/00; G06Q 30/0282

(57) ABSTRACT

Systems, methods, and apparatuses implementing a sentiment-based analytics management system are provided herein. In some embodiments, an example sentiment-based analytics management system may be configured to determine an administrative operations measure for a provider and generate one or more predictive outputs based at least on the administrative operations measure.

### BACKGROUND

Healthcare technology providers may offer revenue cycle management, payment management, and health information exchange (HIE) solutions to various entities including providers and payors.

Patient experiences with providers in healthcare settings may influence financial performance of the providers in ways that are generally unaccounted for in healthcare technology solutions. For example, many negative online reviews for a provider may result in a loss of clients and negatively impact the provider's financial performance. In particular, negative administrative encounters (e.g., incorrect billing, scheduling errors, and the like) may result in client attrition. Additionally, in some cases, administrative operations may be provided at least partly through third-party healthcare technology providers, making it difficult for providers to identify and resolve operational issues. For example, a majority of patients conduct online searches before making an appointment with a physician and these reviews may have up to five times the impact of traditional marketing techniques. In response to a hypothetical review of a physician's office describing a poor administrative experience related to billing, 45% of respondents looked for a different doctor's office.

Conventional software may include return on investment (ROI) calculators for comparing prospective financial data against internal benchmarks. However, such software may fail to quantify or measure the impact of administrative operations (e.g., back-office operations including billing, scheduling, and the like) on the overall financial performance of a given provider. Additionally, existing systems do not incorporate consumer-related sentiment analytics. Accordingly, conventional approaches may contribute to missed revenue, dissatisfied customers, and potential performance liability risk.

Therefore, systems and methods are desired that overcome challenges in the art, some of which are described above. In particular, a sentiment-based analytics management system that is configured to process data from multiple sources and generate predictive outputs that can be used to optimize administrative functions is desired.

### SUMMARY

Embodiments of the present disclosure address challenges relating to correlating data from multiple sources and identifying and exploiting statistical relationships in various fields, including healthcare. Systems in accordance with the present disclosure can be configured to analyze data associated with a plurality of entities (e.g., providers, payors, claims processors, and/or the like) in a revenue cycle management (RCM) environment and generate data analytics/outputs, predictive outputs, and/or the like.

By utilizing some or all of the innovative techniques disclosed herein for performing predictive data analysis steps/operations, various embodiments of the present invention increase efficiency and accuracy of data storage operations, data retrieval operations, and/or query processing operations across various data storage systems, such as various data storage systems that are part of client-server data storage architectures.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1A is an illustration of an exemplary system that can be used to generate predictive outputs and/or user interface data, in accordance with certain embodiments of the present disclosure;

FIG. 1B is an illustration of an exemplary computing device for implementing a sentiment-based analytics management system, in accordance with certain embodiments of the present disclosure;

FIG. 2 is an illustration of another example system that can be used to generate predictive outputs and/or user interface data, in accordance with certain embodiments of the present disclosure;

FIG. 3 is a flowchart that illustrates an exemplary method for generating one or more predictive output(s) and/or user interface data based on analysis of back-office operations, provider entity information, and patient entity information, in accordance with certain embodiments of the present disclosure;

FIG. 4A, FIG. 4B, FIG. 4C, FIG. 4D, FIG. 4E, and FIG. 4F are schematic diagrams depicting operational examples of user interfaces, in accordance with certain embodiments of the present disclosure; and

FIG. 5 shows an example computing environment in which example embodiments may be implemented.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes-from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and to the Figures and their previous and following description.

As will be appreciated by one skilled in the art, the methods and systems may take the form of an entirely hardware embodiment, an entirely software embodiment, or an embodiment combining software and hardware aspects. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general-purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

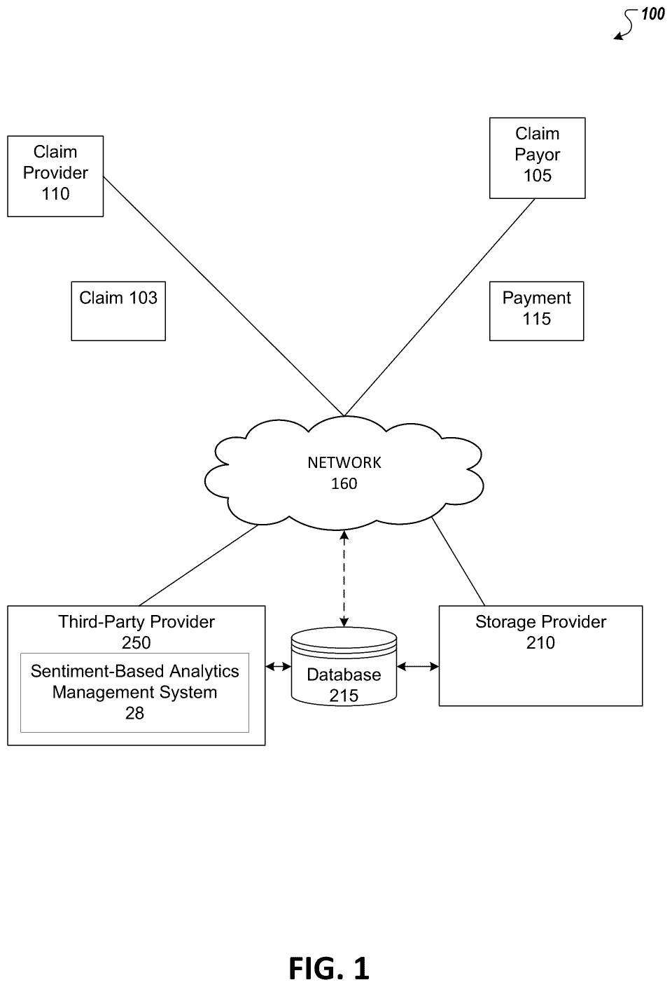

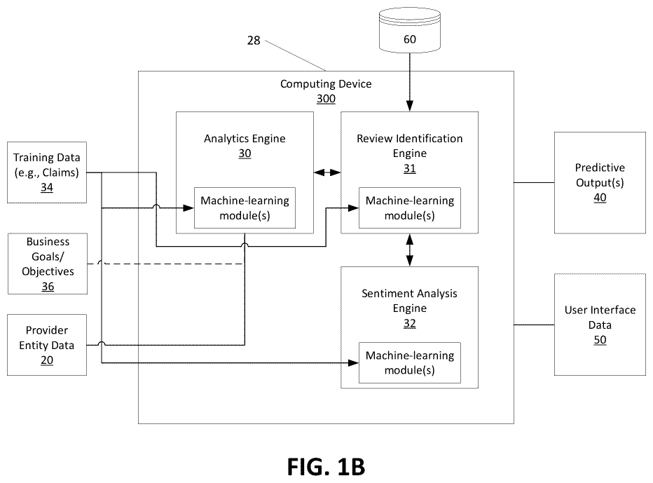

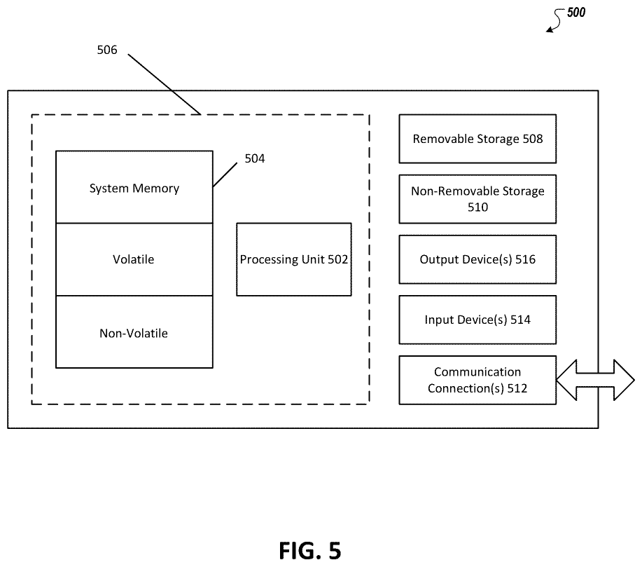

FIG. 1A is an example environment implementing a sentiment-based analytics management system 28 in accordance with certain embodiments of the present disclosure. In various embodiments, the sentiment-based analytics management system 28 can be configured to analyze data associated with one or more entities (e.g., claim providers, claim payors, claims processors, and/or the like) in a revenue cycle management (RCM) environment and generate data analytics/outputs, predictive outputs, and/or the like that can be used to estimate or measure performance or productivity in accordance with business goals or objectives, facilitate optimal allocation of resources, and the like. The sentiment-based analytics management system 28 may be implemented using one or more general purpose computing devices such as the computing device 500 illustrated in FIG. 5.

As shown, the environment 100 may include one or more claim providers 110, one or more claim payors 105, one or more third-party providers 250 (e.g., healthcare technology solutions providers), and one or more storage providers 210 in communication through a network 160. The network 160 may include a combination of private networks (e.g., LANs) and public networks (e.g., the Internet). Each of the one or more claim providers 110 and the one or more claim payors 105 may be partially implemented by one or more general purpose computing devices such as the computing device 500 illustrated in FIG. 5.

The claim provider 110 may be a medical provider or any other entity that provides claims 103 to one or more claim payors 105. The claims 103 may be insurance claims, requests for payment for healthcare services rendered by the claim provider 110, and in some embodiments, claims 103 related to medical services provided to a patient by the claim provider 110 or another entity. In this light, a claim provider 110 may be a physician, technician, nurse, healthcare worker, medical professional, dentist, orthodontist, optometrist, ophthalmologist, and the like. To provide for efficient storage, preserve the privacy of patients associated with the claims 103, and/or healthcare technology solutions, the environment 100 may utilize one or more storage providers 210 and/or one or more third-party providers 250.

The storage provider 210 as described herein includes any entity that provides document storage services and includes dropbox storage providers (“DSP”) and/or cloud-based document storage providers. The storage provider 210 may store the claim 103 in a database 215 (e.g., encrypted data storage). Example storage providers include iCloud, Google Drive, Box, and OneDrive. Each storage provider 210 may expose an application programming interface (API) through which the claim providers 110 and claim payors 105 may write and read documents (e.g., claims 103) from the storage providers 210.

The claim payor 105 may include insurance companies, government entities, or any other entity that may process payments 115 and/or evaluate claims 103 on behalf of patients or other entities. In various embodiments, the third-party provider 250 may provide administrative operations (e.g., back-office or processing services) to claim providers 110. By way of example, a patient may receive medical services from a claim provider 110 (e.g., physician) that results in generation of one or more claims 103. Each claim 103 can be associated with one or more related or unrelated claim providers 110. The claim provider 110 may outsource the processing of certain aspects of the one or more claims 103 to the third-party provider 250 (e.g., claims, processor, clearinghouse, or the like).

Generally, the claims 103 are electronically transmitted over the network 160 to the third-party provider 250 (e.g., claims processor) in a standard electronic format (e.g., In the United States this may be the ANSI ASC X12N 837 format, incorporated by reference), though equivalents and other such formats are contemplated within the scope of this disclosure. The third-party provider 250 (e.g., claims processor) receives the claims 103 from the claim providers 110 and reviews them for completeness, accuracy, containing the correct codes (e.g., Current Procedural Terminology (CPTTM) codes) for the services performed by the claim provider 110, and the like. Incomplete claims and/or claims that appear to be incorrect (i.e., returned claims) are typically automatically and electronically returned to the claim provider 110 to be corrected and re-submitted. Complete and accurate claims 103 are forwarded on to a claim payor 105 (e.g., an insurance company, a governmental entity, and the like). In turn, the claim payor 105 provides a payment 115 to the claim provider 110 in accordance with an agreement between the claim provider 110 and the claim payor 105. The claim payor 105 may also provide feedback to the third-party provider 250 (e.g., claims processor) that indicates how a claim 103 forwarded to the claim payor 105 was handled.

As depicted in FIG. 1A, the third-party provider 250 comprises a sentiment-based analytics management system 28, described in more detail herein. The third-party provider 250 may analyze and process provider entity data (e.g., claims 103) and output user interface data such as data that can be used by the claim provider 110 to set and monitor business goals and objectives. For example, the third-party provider 250 can track key performance indicators (KPIs) of the claim provider 110 that may be used for and/or used to set goals and objectives include throughput (beginning balance+inflows−ending balance); cost to collect; productivity; days in inventory; average production wage rate; Accounts

Receivable (AR); net collection rate; yield; and the like. In some embodiments, the third-party provider 250 may analyze such information in addition to provider entity information (e.g., geographic location, payer mix, and the like) and may generate a comparative analysis for the provider entity against similar provider entities. As described in more detail herein, the third-party provider 250/sentiment-based analytics management system 28 can generate predictive outputs relating to predicted ROI, workflows, resource allocation (e.g., staffing requirements), administrative or back-office related recommendations and other recommendations that can be implemented to improve financial performance of the claim provider 110.

Referring now to FIG. 1B, an example sentiment-based analytics management system 28 in accordance with certain embodiments of the present disclosure is depicted. As shown, the sentiment-based analytics management system 28 is embodied as a computing device 301 that further comprises an analytics engine 30, a review identification engine 31, and a sentiment analysis engine 32. In some embodiments, each of the analytics engine 30, review identification engine 31, and the sentiment analysis engine 32 may be separate or remote from the computing device 301.

In some embodiments, the analytics engine 30 is configured to process provider entity data 20 (such as, but not limited to, claims), business goals/objectives 36, and/or the like that can be used to generate predictive outputs 40 and/or user interface data 50. For example, the analytics engine 30 can be configured to analyze data associated with one or more entities (e.g., providers, payors, claims processors, and/or the like) in a revenue cycle management (RCM) environment and generate data analytics/outputs that can be used to estimate or measure performance or productivity in accordance with business goals or objectives. In some implementations, the analytics engine 30 can process data including in-process claim errors, aggregate statistics regarding client performance in relation to the claims, and output comparative data (e.g., benchmarks) for similar clients.

In some embodiments, the review identification engine 31 is configured to obtain and process review information from one or more online review repositories 60 (e.g., Google, Yelp). For example, the review identification engine 31 may be configured to extract administrative information (e.g., back-office information) from review information associated with a provider entity and correlate (e.g., map) the administrative information with provider data analytics. Administrative information may be or comprise portions of review information that relate to scheduling, counseling, billing, or the like. In some embodiments, the review identification engine 31 may operate in conjunction with a sentiment analysis engine 32. In various implementations, the sentiment analysis engine 32 may be separate from or incorporated with the review identification engine 31. In some embodiments, the sentiment analysis engine 32 may be embodied as a cloud service. It should be understood that embodiments of the present disclosure using cloud services can use any number of cloud-based components or non-cloud based components to perform the processes described herein. In some implementations, the review identification engine 31 may be configured to process obtained review data and provide at least a portion of the review data and/or an analysis associated with the review data to a provider (e.g., customer or potential customer).

The sentiment analysis engine 32 may be configured to identify (e.g., extract) portions of review information that relate to administrative operations. The sentiment analysis engine 32 may process textual data from one or more online review repositories 60 using natural language processing operations. In some examples, the sentiment analysis engine 32 can determine which reviews include text that is relevant to billing processes or operations and determine an overall sentiment (e.g., positive, negative, or neutral) for those reviews. Additionally and/or alternatively, the sentiment analysis engine 32 can determine a sentiment for all reviews associated with a provider and use either determination to compare a given provider to similar providers. In some embodiments, the sentiment analysis engine 32 can process textual data using rules-based grammar standards, statistical classifiers, by calling a cloud or web service for Natural Language Understanding (NLU) processing, and/or any other method or combination of methods for extracting meaning from transcribed text. In some embodiments, the sentiment analysis engine 32 is configured to generate a measure or value describing an inferred determination relating to administrative related operations/performance of a provider entity. By way of example, the sentiment analysis engine 32 may output a score (e.g., between 0 and 1 or between 0% and 100%) where a high or above-threshold score indicates that a given provider entity performs well with respect to administrative operations. The sentiment analysis engine 32 may determine whether an administrative operations measure or score satisfies, meets, or exceeds a predetermined value or threshold (e.g., 75%) where an above-threshold value indicates that the provider entity performs well with respect to administrative operations. The administrative operations measure can be used to determine a predicted ROI or other sales-related metric for the provider entity. In some examples, the third-party provider 250/sentiment-based analytics management system 28 may determine (e.g., in an instance in which the administrative operations measure fails to satisfy, meet or exceed the predetermined value or threshold) one or more suggested actions for improving the administrative operations measure. Examples of suggested actions may include modifying or optimizing one or more administrative processes, changing or implementing a healthcare technology package or the like. In some examples, if the administrative operations measure is a below-threshold value (e.g., a low score), the sentiment-based analytics management system 28 may output a report, for example, including operational values and analytics for similar customers. In some implementations, the report can include predicted outcomes associated with recommended services/software or performance outcomes for similar customers that have implemented such services or software.

In some embodiments, the third-party provider 250/sentiment-based analytics management system 28 comprises a computer-implemented artificial intelligence-enabled engine. The term “artificial intelligence” is defined herein to include any technique that enables one or more computing devices or computing systems (i.e., a machine) to mimic human intelligence. AI includes, but is not limited to, knowledge bases, machine-learning, representation learning, and deep learning. The term “machine-learning” is defined herein to be a subset of AI that enables a machine to acquire knowledge by extracting patterns from raw data. Machine-learning techniques include, but are not limited to, logistic regression, support vector machines (SVMs), decision trees (including randomized decision forests), Naïve Bayes classifiers, AutoRegressive Integrated Moving Average (ARIMA) machine-learning algorithms, and artificial neural networks. The term “representation learning” is defined herein to be a subset of machine-learning that enables a machine to automatically discover representations needed for feature detection, prediction, or classification from raw data. Representation learning techniques include, but are not limited to, autoencoders. The term “deep learning” is defined herein to be a subset of machine-learning that enables a machine to automatically discover representations needed for feature detection, prediction, classification, etc. using layers of processing. Deep learning techniques include, but are not limited to, artificial neural network (including deep nets, long short-term memory (LSTM) recurrent neural network (RNN) architecture), or multilayer perceptron (MLP). Machine-learning models include supervised, semi-supervised, and unsupervised learning models. In a supervised learning model, the model learns a function that maps an input (also known as feature or features) to an output (also known as a target or target) during training with a labeled data set (or dataset). In an unsupervised learning model, the model learns a function that maps an input (also known as feature or features) to an output during training with an unlabeled data set. In a semi-supervised model, the model learns a function that maps an input (also known as feature or features) to an output (also known as a target or target) during training with both labeled and unlabeled data.

Each of the analytics engine 30, the review identification engine 31, and the sentiment analysis engine 32 may include a machine-learning (e.g., training) module and a trained AI module that can be used for processing data (e.g., in order to generate predictive outputs 40). Accordingly, each of the analytics engine 30, the review identification engine 31, and the sentiment analysis engine 32 may use training data and/or claims data 34 for training a machine learning module.

The sentiment-based analytics management system described herein may comprise all or part of an artificial neural network (ANN). An ANN is a computing system including a plurality of interconnected neurons (e.g., also referred to as “nodes”). This disclosure contemplates that the nodes can be implemented using a computing device (e.g., a processing unit and memory as described herein), such as computing device 500 described herein. The nodes can be arranged in a plurality of layers such as input layer, output layer, and optionally one or more hidden layers. An ANN having hidden layers can be referred to as deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tan H, or rectified linear unit (ReLU) function), and provide an output in accordance with the activation function. Additionally, each node is associated with a respective weight. ANNs are trained with a dataset to maximize or minimize an objective function (e.g., the business goals and objectives). In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include, but are not limited to, backpropagation. It should be understood that an artificial neural network is provided only as an example machine-learning model. This disclosure contemplates that the machine-learning model can be any supervised learning model, semi-supervised learning model, or unsupervised learning model. Optionally, the machine-learning model is a deep learning model. Machine-learning models are known in the art and are therefore not described in further detail herein.

A convolutional neural network (CNN) is a type of deep neural network that can be applied, for example, to non-linear workflow prediction applications, such as those described herein. Unlike a traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similar to traditional neural networks. GCNNs are CNNs that have been adapted to work on structured datasets such as graphs.

Other supervised learning models that may be utilized according to embodiments described herein include a logistic regression (LR) classifier, a Naïve Bayes' (NB) classifier, a k-NN classifier, a majority voting ensemble, and the like.

By way of example, the analytics engine 30 may comprise a machine-learning module that is trained using training data 34. The training data 34 can comprise claim information describing services provided to one or more patients, information associated with the one or more providers that provided the services to the one or more patients, information associated with the payors of the medical claims including feedback from the claim payors on the handling of specific claims by the claim payor. In some instances, the training data 34 may at least be partially comprised of historical data extracted from past claims. The training data 34 may also include exemplary business goals and objectives, such as maximizing profit, leaving no or minimal backlog, minimizing expenses, keeping average days in accounts receivable (DAR) below a threshold, and the like. The analytics engine 30/machine-learning module may be further configured to identify individual independent variables that are used by the trained machine-learning module to make predictions, which may be considered a dependent variable. For example, the training data 34 may be generally unprocessed or formatted and include extra information in addition to medical claim information, provider information, and payor information. For example, the medical claim data may include account codes, codes associated with the services performed by the provider, business address information, and the like, which can be filtered out by the analytics engine 30/machine-learning module. The features extracted from the training data 34 may be called attributes and the number of features may be called the dimension. The analytics engine 30/machine-learning module may further be configured to assign defined labels to the training data 34 and to the generated predictions to ensure a consistent naming convention for both the input features and the predicted outputs. The machine-learning module 40 processes both the featured training data 34, including the labels, and may be configured to test numerous functions to establish a quantitative relationship between the featured and labeled input data and the predicted outputs. The analytics engine 30/machine-learning module may use modeling techniques, as described herein, to evaluate the effects of various input data features on the predicted outputs. These effects may then be used to tune and refine the quantitative relationship between the featured and labeled input data and the predicted outputs. The tuned and refined quantitative relationship between the featured and labeled input data generated by the analytics engine 30/machine-learning module is output for use in the trained machine-learning module.

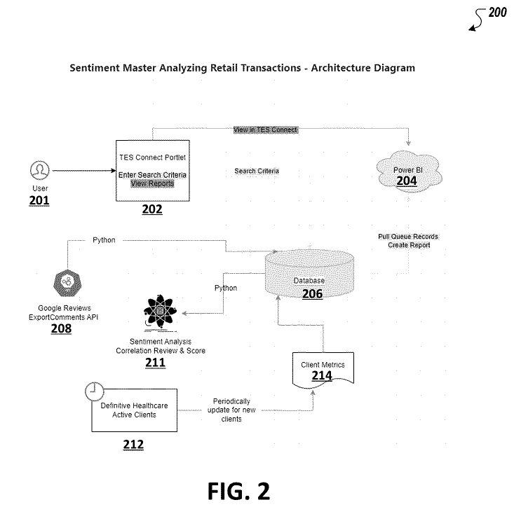

Referring now to FIG. 2, a schematic diagram depicting an example system 200 for implementing certain embodiments of the present disclosure is provided. As depicted, the system 200 includes a client computing entity 202, a cloud-based business intelligence platform 204 (e.g., Power Bi), and one or more databases 206. The client computing entity 202 may be in electronic communication with a database 206 and a cloud-based business intelligence platform 204.

In various implementations, a user 201 (e.g., provider entity) may access the system 200 through an API via the client computing entity 202. In some implementations, the user 201 may access services (e.g., healthcare technology solutions) by obtaining a subscription or license. In some embodiments, such services may be incorporated with claims processing services (e.g., provided by a third-party provider 250 implementing a sentiment-based analytics management system 28 as described above in conjunction with FIG. 1A).

As shown, the database 206 can store review information 208 (e.g., obtained from a review provider API, such as but not limited to Google and Yelp), provider entity information 212, and client metrics 214 (e.g., provider entity information) that can be used to generate predictive outputs, such as but not limited to, an administrative operations measure or score 211 (e.g., sentiment analysis correlation review based at least on analysis of the review information 208 and client metrics 214).

As further depicted in FIG. 2, the cloud-based business intelligence platform 204 can be configured to process at least a portion of the review information 208, provider entity information 212, and the client metrics 214 (e.g., transform, clean, or the like) and generate user interface data such as visualizations (e.g., charts, graphs, or the like) for presentation or display by the client computing entity 202.

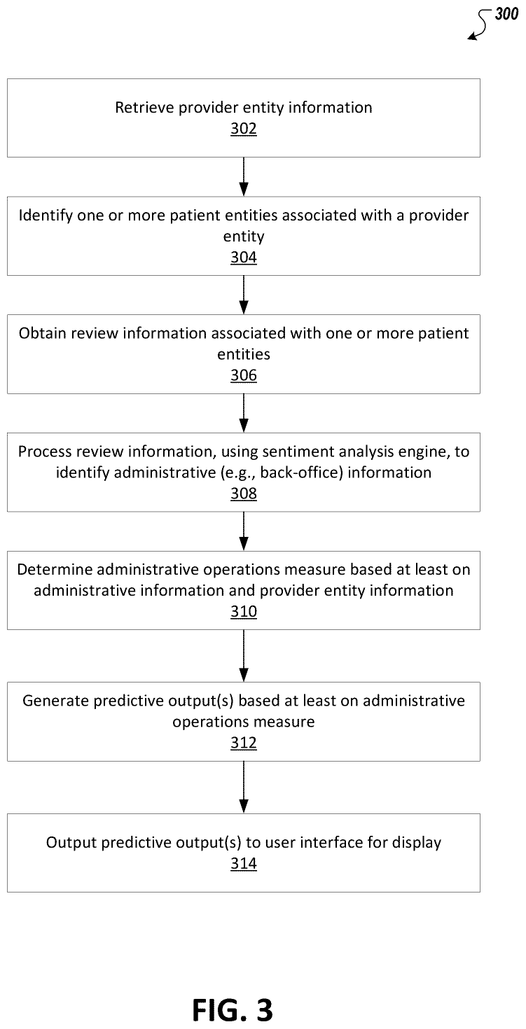

FIG. 3 is a flowchart diagram that illustrates an exemplary method 300 that can lead to generating and outputting one or more predictive outputs to a user interface.

Beginning at step/operation 302, the sentiment-based analytics management system (such as, but not limited to, the sentiment-based analytics management system 28 described above in connection with FIG. 1B) retrieves provider entity information. The provider entity information may comprise historical data, financial data, medical data, client related information, and/or the like. In some embodiments, step/operation 302 includes generating and/or training the sentiment-based analytics management system.

Subsequent to step/operation 302, the method 300 proceeds to step/operation 304. At step/operation 304, the sentiment-based analytics management system identifies one or more patient entities associated with a provider entity. In some embodiments, identifying the one or more patient entities comprises analyzing the provider entity information to identify one or more patients that have received medical services from the provider entity within a defined time period and/or at a particular geographic location.

Subsequent to step/operation 304, the method 300 proceeds to step/operation 306. At step/operation 306, the sentiment-based analytics management system obtains review information associated with the one or more patient entities. Each patient entity may be associated with a patient profile comprising member information/data, member features, and/or similar words used herein interchangeably that can be associated with a given member identifier for a patient/individual, claim(s), and/or the like. In some embodiments, a patient profile may include age, gender, known health conditions, home location, medical history, claim history, a member identifier (ID), and/or the like. In some examples, the sentiment-based analytics management system can aggregate patient entity information and identify patient trends and/or patterns relating to patient retention. In some embodiments, step/operation 304 comprises mapping the one or more patient entities to stored data in one or more online review repositories (e.g., Google, Yelp, or the like). In some embodiments, the sentiment-based analytics management system scrapes data from the one or more online review repositories. In some implementations, the review information may be identified by aggregating review information that satisfies one or more parameters, including but not limited to, geographic data (e.g., home addresses, billing addresses, zip codes), review type (e.g., healthcare, medical), and the like. For example, reviews may be attributed to a provider entity by associating a physical geographical location of the review (e.g., based on analysis of metadata, timestamp data) and the provider entity's geographical information.

Subsequent to step/operation 306, the method 300 proceeds to step/operation 308. At step/operation 308, the sentiment-based analytics management system processes the review information (e.g., obtained from one or more online review repositories), using a sentiment analysis engine (such as, but not limited to, sentiment analysis engine 32 described above in connection with FIG. 1B) to identify administrative information. Administrative information may be or comprise portions of review information that relate to back-office operations such as scheduling, counseling, billing, or the like. The sentiment-based analytics management system can identify administrative information using keyword-based extraction techniques (e.g., identifying particular terms, such as but not limited to, “scheduling”, “counseling”, “billing”, and the like).

Subsequent to step/operation 308, the method 300 proceeds to step/operation 310. At step/operation 310, the sentiment-based analytics management system determines an administrative operations measure based at least on the administrative information and the provider entity information. For example, the sentiment-based analytics management system can compare review trends with trends identified in the patient entity information (e.g., patient retention information and/or net new patients). The sentiment-based analytics management system can generate and/or utilize a model or mathematical expression that correlates revenue-related values with an administrative operations measure. The sentiment-based analytics management system may generate the example model by analyzing administrative information, provider entity information, and/or patient entity information for a plurality of providers. The sentiment-based analytics management system may use one or more machine learning model models (e.g., a neural network, deep learning model, CNN or the like) to generate metrics relating to provider performance. In some embodiments, the sentiment-based analytics management system can generate and/or utilize a function that estimates retained and/or new revenue based on the administrative operations measure. For example, a 70% administrative operations is associated with a 3% projected increase in revenue, and an 80% administrative operations measure is associated with a 6% projected increase in revenue. In some embodiments, the sentiment-based analytics management system may use a linear regression function to correlate administrative operations perception with revenue for similar clients.

Subsequent to step/operation 310, the method 300 proceeds to step/operation 312. At step/operation 312, the sentiment-based analytics management system generates one or more predictive outputs based at least on the administrative operations measure. By way of example, the sentiment-based analytics management system can trigger generation (e.g., by client computing entity 202) of user interface data (e.g., messages, data objects and/or the like) corresponding with predictive outputs. The client computing entity 202 may provide the user interface data for presentation by a user computing entity. In some embodiments, the user interface data may be or comprise the administrative operations measure and recommendations corresponding thereto.

In some embodiments, the sentiment-based analytics management system determines the administrative operations measure and/or one or more predictive outputs (e.g., recommendations) based at least on business goals or objectives that can be set by the provider. In some examples, the business goals or objectives can comprise one or more of having no backlog of the claims for review that are not reviewed by the available resources during a defined time period; having DAR for the future inflow of work less than a defined number of days; maximizing revenue; minimizing costs; having a defined amount of throughput during the defined time period; having a cost to collect at or below a threshold; having a measurement of a productivity of staff at, above or below a certain threshold over the defined time period; having days in inventory at, above or below a certain threshold; having an average production wage rate at, above or below a certain threshold; and/or having a net collection rate. Accordingly, in various implementations, a provider can provide business outcomes that can be used to generate recommendations.

Subsequent to step/operation 312, the method 300 proceeds to step/operation 314. At step/operation 314, the sentiment-based analytics management system outputs the predictive output(s) to a user interface for display. The sentiment-based analytics management system may be configured to generate one or more API-based data objects corresponding with at least a portion of the predictive outputs. The sentiment-based analytics management system may provide (e.g., transmit, send) the one or more API-based data objects representing at least a portion of the predictive outputs to an end user interface for display and/or further steps/operations. The predictive outputs may be used to dynamically update the user interface operated by an end user.

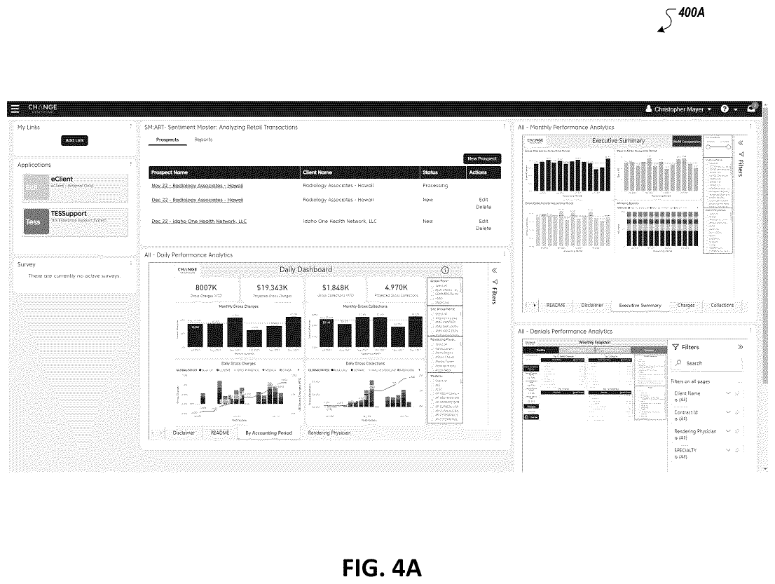

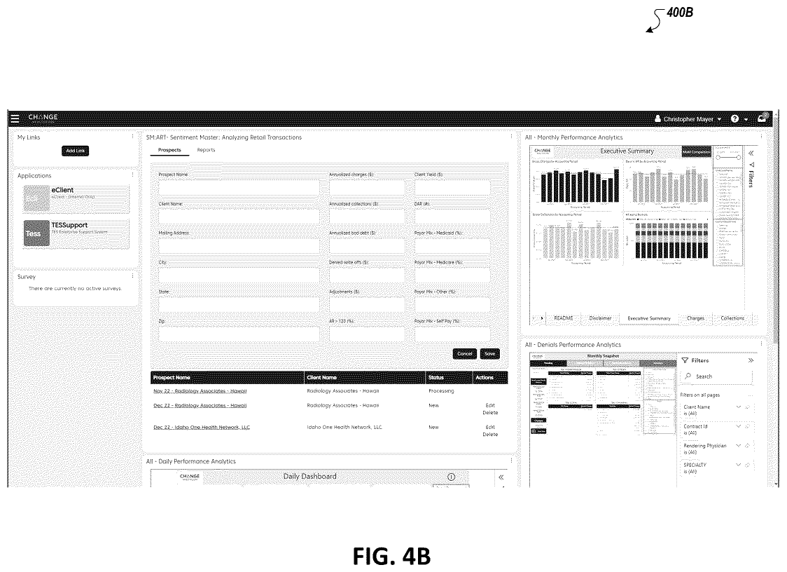

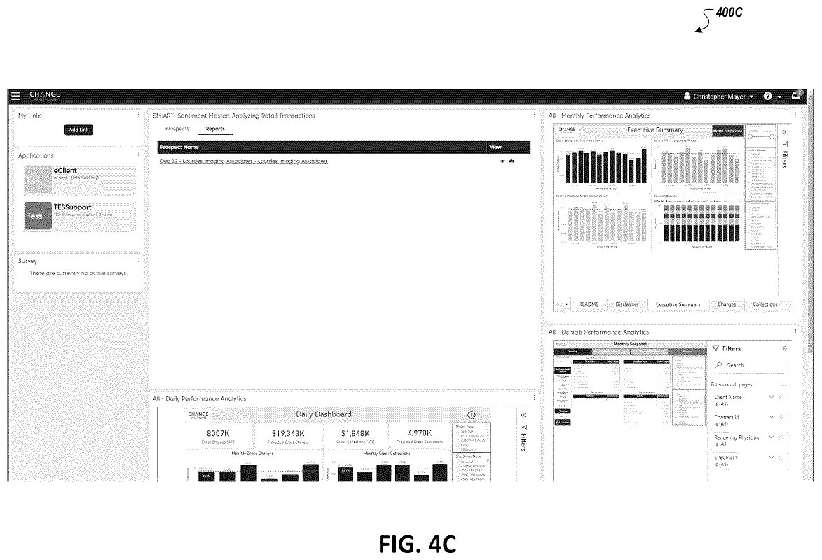

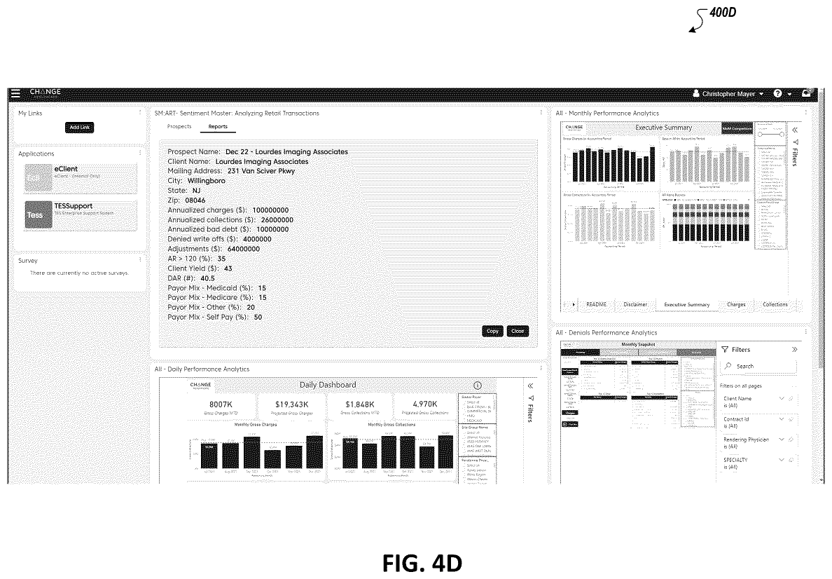

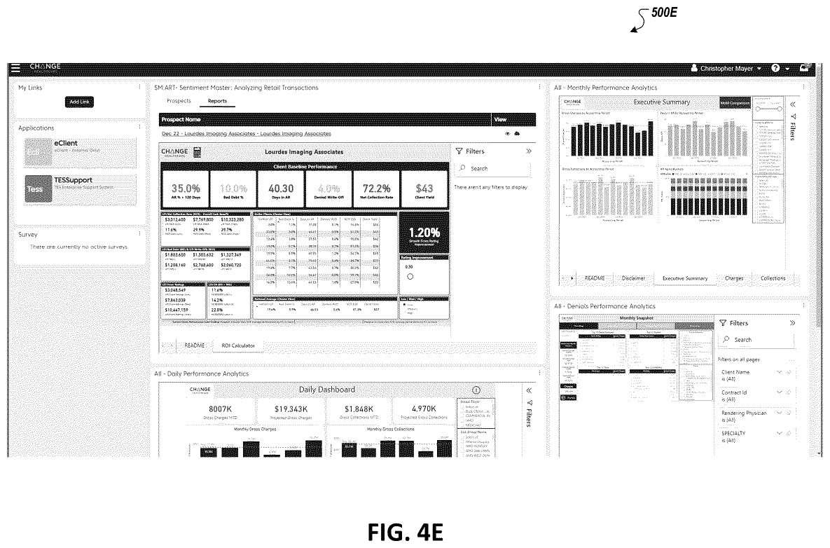

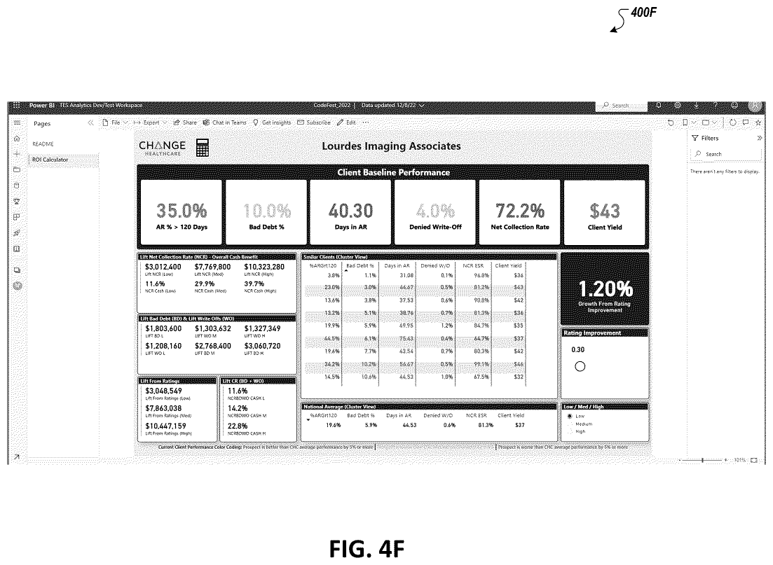

Referring now to FIG. 4A-4F, operational examples depicting user interfaces 400A, 400B, 400C, 400D, 400E, and 400F) that may be generated based at least in part on user interface data which is in turn generated based at least in part on the above-described predictive outputs. A client computing entity (e.g., client computing entity 202) may generate the user interface data and present (e.g., transmit, send, and/or the like) corresponding user interface data for presentation by the user interfaces 400A-F.

As depicted in FIGS. 4A-F, the user interface comprises various user-selectable interface elements for accessing the user interface data. In particular, as shown, a user may engage/select a user-selectable interface element to view performance data associated with a provider entity, and/or provide additional input data/parameters (e.g., business goals and/or objectives) that can be used to generate predictive outputs and/or update user interface data.

Additionally, the user interfaces 400A-F may comprise various additional features and functionalities for accessing, and/or viewing user interface data. The user interfaces 400A-F may also comprise messages to an end-user in the form of banners, headers, notifications, and/or the like. As will be recognized, the described elements are provided for illustrative purposes and are not to be construed as limiting the dynamically updatable interface in any way.

FIG. 5 shows an example computing environment in which example embodiments and aspects may be implemented. The computing device environment is only one example of a suitable computing environment and is not intended to suggest any limitation as to the scope of use or functionality.

Numerous other general purpose or special purpose computing devices environments or configurations may be used. Examples of well-known computing devices, environments, and/or configurations that may be suitable for use include, but are not limited to, personal computers, server computers, handheld or laptop devices, multiprocessor systems, cloud-based systems, microprocessor-based systems, network personal computers (PCs), minicomputers, mainframe computers, embedded systems, distributed computing environments that include any of the above systems or devices, and the like. The computing environment may include a cloud-based computing environment.

Computer-executable instructions, such as program modules, being executed by a computer may be used. Generally, program modules include routines, programs, objects, components, data structures, etc. that perform particular tasks or implement particular abstract data types. Distributed computing environments may be used where tasks are performed by remote processing devices that are linked through a communications network or other data transmission medium. In a distributed computing environment, program modules and other data may be located in both local and remote computer storage media including memory storage devices.

With reference to FIG. 5, an example system for implementing aspects described herein includes a computing device, such as computing device 500. In its most basic configuration, computing device 500 typically includes at least one processing unit 502 and memory 504. Depending on the exact configuration and type of computing device, memory 504 may be volatile (such as random access memory (RAM)), non-volatile (such as read-only memory (ROM), flash memory, etc.), or some combination of the two. This most basic configuration is illustrated in FIG. 5 by dashed line 506.

Computing device 500 may have additional features/functionality. For example, computing device 500 may include additional storage (removable and/or non-removable) including, but not limited to, magnetic or optical disks or tape. Such additional storage is illustrated in FIG. 5 by removable storage 508 and non-removable storage 510.

Computing device 500 typically includes a variety of computer readable media. Computer readable media can be any available media that can be accessed by the device 500 and includes both volatile and non-volatile media, removable and non-removable media.

Computer storage media include volatile and non-volatile, and removable and non-removable media implemented in any method or technology for storage of information such as computer readable instructions, data structures, program modules or other data. Memory 504, removable storage 508, and non-removable storage 510 are all examples of computer storage media. Computer storage media include, but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information, and which can be accessed by computing device 500. Any such computer storage media may be part of computing device 500.

Computing device 500 may contain communication connection(s) 512 that allow the device to communicate with other devices. Computing device 500 may also have input device(s) 514 such as a keyboard, mouse, pen, voice input device, touch input device, etc. Output device(s) 516 such as a display, speakers, printer, etc. may also be included. All these devices are well known in the art and need not be discussed at length here.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited, but rather may be implemented in connection with any computing environment, such as a network or distributed computing environment. Still further, aspects of the presently disclosed subject matter may be implemented in or across a plurality of processing chips or devices, and storage may similarly be effected across a plurality of devices. Such devices might include personal computers, network servers, and handheld devices, for example.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed:

1. A sentiment-based analytics management system comprising:
at least one computing device; and
a memory storing computer-readable instructions that when executed by the at least one computing device cause the at least one computing device to:
identify one or more patient entities associated with a provider entity;
obtain review information associated with the one or more patient entities;
process the review information, using a sentiment analysis engine, to identify administrative information from the review information;
determine an administrative operations measure based at least on the administrative information and provider entity information;
generate one or more predictive outputs based at least on the administrative operations measure; and
output at least one predictive output to a user interface for display.

2. The sentiment-based analytics management system of claim 1, wherein processing the review information to identify administrative information comprises using a keyword-based extraction operation.

3. The sentiment-based analytics management system of claim 1, wherein processing the review information to identify administrative information comprises processing textual data from one or more online review repositories using natural language processing operations.

4. The sentiment-based analytics management system of claim 1, wherein the administrative operations measure comprises a value or score an inferred determination value describing an inferred determination relating to administrative related operations of the provider entity.

5. The sentiment-based analytics management system of claim 1, wherein the computer-readable instructions further comprise instructions that when executed by the at least one computing device cause the at least one computing device to:
determine whether the administrative operations measure satisfies, meets, or exceeds a predetermined threshold, where an above-threshold value indicates that the provider entity performs well with respect to administrative operations.

6. The sentiment-based analytics management system of claim 1, wherein the one or more predictive outputs comprises a predicted Return on Investment (ROI).

7. The sentiment-based analytics management system of claim 1, further comprising computer-readable instructions that when executed by the at least one computing device cause the at least one computing device to train the sentiment analysis engine.

8. The sentiment-based analytics management system of claim 1, wherein one or more of the administrative operations measure and the one or more predictive outputs are determined based at least on one or more business goals and objectives.

9. The sentiment-based analytics management system of claim 8, wherein the one or more business goals and objectives include one or more of having no backlog of the claims for review that are not reviewed by available resources during a defined time period; having an average days in accounts receivable (DAR) for a future inflow of work less than a defined number of days; maximizing revenue; minimizing costs; having a defined amount of throughput during the defined time period; having a cost to collect at or below a threshold; having a measurement of a productivity of staff at, above or below a certain threshold over the defined time period; having days in inventory at, above or below a certain threshold; having an average production wage rate at, above or below a certain threshold; and/or having a net collection rate.

10. A computer-implemented method comprising:
identifying, by one or more processors, one or more patient entities associated with a provider entity;
obtaining, by the one or more processors, review information associated with the one or more patient entities;
processing, by the one or more processors, the review information, using a sentiment analysis engine, to identify administrative information from the review information;
determining, by the one or more processors, an administrative operations measure based at least on the administrative information and provider entity information;
generating, by the one or more processors, one or more predictive outputs based at least on the administrative operations measure; and
outputting, by the one or more processors, at least one predictive output to a user interface for display.

11. The computer-implemented method of claim 10, wherein processing the review information to identify administrative information comprises using a keyword-based extraction operation.

12. The computer-implemented method of claim 10, wherein processing the review information to identify administrative information comprises processing textual data from one or more online review repositories using natural language processing operations.

13. The computer-implemented method of claim 10, wherein the administrative operations measure comprises a value or score an inferred determination value describing an inferred determination relating to administrative related operations of the provider entity.

14. The computer-implemented method of claim 10, further comprising
determining, by the one or more processors, whether the administrative operations measure satisfies, meets, or exceeds a predetermined threshold, where an above-threshold value indicates that the provider entity performs well with respect to administrative operations.

15. The computer-implemented method of claim 10, wherein the one or more predictive outputs comprises a predicted Return on Investment (ROI).

16. The computer-implemented method of claim 10, further comprising:
training, by the one or more processors, the sentiment analysis engine.

17. The computer-implemented method of claim 10, wherein one or more of the administrative operations measure and the one or more predictive outputs are determined based at least on one or more business goals and objectives.

18. The computer-implemented method of claim 17, wherein the one or more business goals and objectives include one or more of having no backlog of the claims for review that are not reviewed by available resources during a defined time period; having an average days in accounts receivable (DAR) for a future inflow of work less than a defined number of days; maximizing revenue; minimizing costs; having a defined amount of throughput during the defined time period; having a cost to collect at or below a threshold; having a measurement of a productivity of staff at, above or below a certain threshold over the defined time period; having days in inventory at, above or below a certain threshold; having an average production wage rate at, above or below a certain threshold; and/or having a net collection rate.

19. A non-transitory computer-readable medium with computer-executable instructions stored thereon that when executed by at least one computing device cause the at least one computing device to:
identify one or more patient entities associated with a provider entity;
obtain review information associated with the one or more patient entities;
process the review information, using a sentiment analysis engine, to identify administrative information from the review information;
determine an administrative operations measure based at least on the administrative information and provider entity information;
generate one or more predictive outputs based at least on the administrative operations measure; and
output at least one predictive output to a user interface for display.

20. The non-transitory computer-readable medium of claim 19, wherein processing the review information to identify administrative information comprises using a keyword-based extraction operation.
